(19) United States
(12) Patent Application Publication
(10) Pub. No.: US 2003/0228270 A1
(43) Pub. Date: Dec. 11, 2003

(54) Foaming clay cleanser composition

(76) Inventors: Erika Tazberik, Huntington Beach, CA (US); Snehal Shah, Cerritos, CA (US)

Correspondence Address: PHILIP S. JOHNSON, JOHNSON & JOHNSON, ONE JOHNSON & JOHNSON PLAZA, NEW BRUNSWICK, NJ, 08933-7003, US

(21) Appl. No.: 10/166,455

(22) Filed: Jun. 10, 2002

(51) Int. Cl. A61K 7/075; A61K 7/08

(52) U.S. Cl. 424/70.22

(57) ABSTRACT

The present invention relates to a product containing a composition wherein the composition contains at least one clay, at least one foaming surfactant, and water.

### FIELD OF THE INVENTION

The present invention relates to a foaming cleanser composition containing clay.

### BACKGROUND OF THE INVENTION

Traditional mask compositions are non-foaming products typically used on the face for intense facial cleansing, treatment, or oil control. Examples of such masks include Neutrogena® Oil-absorbing Acne Mask and Neutrogena® Pore Refining Mask. Such masks are applied to the skin and, after a specific drying time, are rinsed off with water. The removal of such masks, however, can be difficult.

Masks are often only applied periodically, two or three times of week. Masks are also generally applied after first cleansing the skin. Thus, users are required to purchase separate products to cleanse the skin prior to application of the mask as for use on days when they do not wish to apply the mask.

Unlike traditional masks, the composition of present invention can be used both as a facial cleanser as well as a mask. The composition of the present invention, thus, can be used everyday without the need to purchase an additional cleanser. The surfactant in the product also facilitates the removal of the mask from the skin.

### SUMMARY OF THE INVENTION

In one aspect, the present invention relates to a product containing a composition wherein the composition contains at least one clay, at least one foaming surfactant, and water. In one embodiment, the product includes instructions directing the user to apply the composition to wet skin and then to remove the composition from the skin.

In another aspect, the present invention relates to a method of promoting a product containing the above composition wherein the method includes directing the user to apply the composition to wet skin and then remove said composition from said skin.

In another aspect, the present invention relates to a method of cleansing the skin including: wetting the skin; (ii) applying a composition containing at least one clay, at least one foaming surfactant, and water; and (iii) removing the composition from the skin.

Other features and advantages of the present invention will be apparent from the detailed description of the invention and from the claims

### DETAILED DESCRIPTION OF THE INVENTION

It is believed that one skilled in the art can, based upon the description herein, utilize the present invention to its fullest extent. The following specific embodiments are to be construed as merely illustrative, and not limitative of the remainder of the disclosure in any way whatsoever.

Unless defined otherwise, all technical and scientific terms used herein have the same meaning as commonly understood by one of ordinary skill in the art to which the invention belongs. Also, all publications, patent applications, patents, and other references mentioned herein are incorporated by reference.

What is meant by a “product” is a product in finished packaged form. In one embodiment, the package is a container such as pumps, jars, or tubes containing the composition. The product may further contain additional packaging such as a plastic or cardboard box for storing such container. In one embodiment, the product contains instructions directing the user to use the composition as a cleanser. For example, (i) to apply the composition to wet skin (e.g., to apply the composition to skin that is already wet or to apply water in conjunction with applying the composition) and (ii) then to remove the composition from the skin (e.g., to rinse the composition from the face with water). Such instructions may instruct the user to apply the composition by messaging or rubbing the composition into the skin, for example, to form lather prior to rinsing the composition.

In a further embodiment, the instructions rather or as an alternative direct the user to apply the composition as a mask. For example, (i) to apply the composition to dry skin, (ii) to retain the composition on the skin, and (iii) to remove said composition from said skin. In one embodiment, the user is instructed to retain the composition on the skin for a period of time to allow the composition to dry and/or for a period of time of at least about one minute and less than about ten minutes (e.g., such as less than about five minutes).

In one embodiment, the instructions direct the user to apply the composition to the whole body or just to parts of the body such as the face, arms, or legs. Such instructions may be printed on the container, label insert, or on any additional packaging.

What is meant by “promoting” is promoting, advertising, or marketing. Examples of promoting include, but are not limited to, written, visual, or oral statements made on the product or in stores, magazines, newspaper, radio, television, Internet, and the like. Examples of such statements include, but are not limited to, “use as a cleanser,” “use as a daily cleanser,” “use as a mask,” or “use as an intensive mask.”

As used herein, “topical application” means directly laying on or spreading on outer skin using, e.g., by use of the hands or an applicator such as a wipe.

As used herein, “cosmetically-acceptable” means that the cosmetically active agents or inert ingredients which the term describes are suitable for use in contact with tissues (e.g., the skin) without undue toxicity, incompatibility, instability, irritation, allergic response, and the like, commensurate with a reasonable benefit/risk ratio.

As used herein, “safe and effective amount” means an amount of compound or composition sufficient to significantly induce a positive modification in the condition to be regulated or treated, but low enough to avoid serious side effects. The safe and effective amount of the compound or composition will vary with the particular condition being treated, the age and physical condition of the end user, the severity of the condition being treated/prevented, the duration of the treatment, the nature of concurrent therapy, the specific compound or composition employed, the particular cosmetically-acceptable topical carrier utilized, and like factors.

Composition

The composition of the present invention includes one or more foaming surfactants. In one embodiment, the composition produces a foam volume of at least 200 ml (as defined herein in the Foaming Test), preferably, a foam volume of at least 300 ml and most preferably 400 ml.

In one embodiment, the composition has a viscosity of about 50,000 cps to about 150,000 cps measured with a Brookfield viscometer RVD VI+ equipped with a Brookfield Helipath Stand Model D at 25° C. using T-C spindle at 5 rpm. The viscosity is reported as an average of 5 measurements at the given revolution going downwards and 5 measurements at the given revolution going upwards.

The composition of the present invention includes water. In one embodiment, the composition includes about 10 to about 90 percent, by weight, of water, e.g., about 20 to about 80 percent, by weight (such as about 20 to about 50 percent by weight), of water.

Clays

The composition of the present invention includes one or more clays. In one embodiment, the clay is an aluminum silicate. Examples of aluminum silicates include, but are not limited to, bentonite, kaolin, hectorite, montmorillonite, and laponite.

In one embodiment, the composition includes about 5 to about 50 percent, by weight, of said at least one clay, e.g., about 10 to about 50 percent, by weight (such as about 20 to about 40 percent, by weight) of the at least one clay.

Foaming Surfactants

The composition of the present invention includes one or more foaming surfactant(s). What is meant by a foaming surfactant is a surfactant that produces a foam volume of at least 200 ml (as defined herein in the Foaming Test). Examples of foaming surfactants are found on pages 1673-89 of the International Cosmetic Ingredient Dictionary and Handbook (7^{th} Ed., The Cosmetic, Toiletry, and Fragrance Association, Washington, D.C., 1997), hereinafter the ICI Handbook. In one embodiment, the foaming surfactant is an anionic, amphoteric, or nonionic surfactant. Examples of anionic foaming surfactants include, but are not limited to, sodium, magnesium, potassium, or ammonium salts of alkyl acyl taurates, alkyl sulfates, alkyl ether sulfates, olefin sulfonates, acyl isethionates, acyl sarcosinates, alkyl sulfosuccinates, and C₈-C₂₂ fatty acids. Specific examples of anionic surfactants include, but are not limited to, sodium cocoyl sarcosinate, sodium cocoyl isethionate, disodium laureth sulfosuccinate, sodium lauroyl lactylate, sodium laurate, sodium myristate, sodium palmitate, sodium methyl cocoyl taurate, and sodium lauroyl sarcosinate.

Examples of amphoteric and nonionic foaming surfactants include, but are not limited to, sodium cocoampho(di)acetate, sodium laurampho(di)acetate, sodium cocoamphopropionate, cocamidopropyl betaine, alkylpolyglucosides, and poloxamers.

In one embodiment, the composition includes about 0.1 to about 50 percent, by weight, of said at least one foaming surfactant, e.g., about 0.5 to about 30 percent, by weight (such as about 1 to about 15 percent, by weight) of the at least one foaming surfactant.

Anti-Acne Agent

In one embodiment, the composition of the present invention includes one or more antiacne agents. What is meant by an “antiacne agent” is a drug product effective F1 is the treatment of acne. Examples of antiacne agents include, but are not limited to, azelaic acid, clindamycin, adapalene, erythromycin, sodium sulfacetamide, retinoic acid, benzoyl peroxide, sulfur, and salicylic acid.

In one embodiment, the composition includes about 0.1 to about 50 percent, by weight, of said at least one antiacne agents, e.g., about 0.5 to about 30 percent, by weight, such as about 0.5 to about 15 percent, by weight, of the at least one antiacne agent. In one embodiment, the composition further comprises a natural extract to enhance the anti-acne efficacy of the anti-acne agent. Examples of such extracts include, but are not limited to, angelica archangelica root extract, dandelion extract, turmeric extract, and melia azadirachta leaf extract.

Viscosity Increasing Agents

In addition to the clays, the composition may further include one or more additional viscosity increasing agents. Examples of viscosity increasing agents include, but are not limited to, xanthan gum, sclerotium gum, guar gum, locust bean gum, alginates, carageenan, cellulose gum, hydroxymethyl or (ethyl or propyl)cellulose, amorphous silicon dioxide such as hydrated silica or silica, acrylic acid polymers, acrylic/acrylamide polymers, alkylene oxide polymers and esters, PVM/MA decadiene crosspolymers, and trihydroxystearin. Other examples of viscosity increasing agents can be found on pages 1693-97 of the ICI Handbook. The amount of viscosity increasing agent(s) included in the composition will depend on the desired viscosity of the composition.

Colorants

The composition of the present invention may also include one or more colorants. The colorants may be either organic or inorganic colorants. Examples of organic colorants include, but are not limited to, various aromatic colorants including azo, indigoid, triphenylmethane, anthraquinone, and xanthine dyes which are designated as D&C and FD&C colors (e.g., blues, greens, oranges, reds, violet, and yellows). Organic colorants generally consist of insoluble metallic salts of certified color additives, referred to as the Lakes, in particular the Lakes of D&C and FD&C colors. Inorganic colorants include titanium dioxide, iron oxides, ultramarines, chromium, and chromium hydroxide colors. Other colorants are listed on pages 1628-30 of the ICI Handbook. The amount of colorant(s) will depend on the desired color of the composition.

Cooling Agents

In one embodiment, the composition includes one or more cooling agents. What is meant by a “cooling agent” is an ingredient which when topically applied on skin creates a sensation of coolness and freshness. Examples of cooling agents include, but are not limited to, menthol, methyl lactate, menthone glycerin acetal, camphor, peppermint leaf extract, and balm mint leaf extract.

In one embodiment, the composition includes about 0.01 to about 2 percent, by weight, of said at least one cooling agent, e.g., about 0.05 to about 1 percent, by weight, such as about 0.05 to about 0.5 percent, by weight, of the at least one cooling agent.

Skin Conditioning Agents

In one embodiment, the composition includes one or more skin conditioning agents. Examples of skin conditioning agents include, but are not limited to, emollients and humectants such as propylene glycols, glycerin, and polyethylene glycols such as Trideceth-9 and PEG-5 Ethylhexanoate. Other skin conditioning agents are listed on pages 1656-70 of the ICI Handbook.

In one embodiment, the composition includes about 0.01 to about 30 percent, by weight, of said at least one skin conditioning agent, e.g., about 0.1 to about 20 percent, by weight (such as about 0.5 to about 15 percent, by weight) of the at least one skin conditioning agent.

Other Ingredients

The composition may further comprise one or more chelating agents such as disodium EDTA, pH adjusters such as citric acid and sodium citrate, vitamins such as vitamins A, Bs, C, and E, fragrances, and preservatives such as parabens and phenoxyethanol. Examples of such are disclosed on pages 1626, 1639-40, and 1653-55 of the ICI Handbook.

Foaming Test

The follow test is used to determine the foam volume of the test composition (e.g., the foaming surfactant or the composition). One gram of the test composition is added into a beaker with ninety-nine grams of deionized water. The mixture is then mixed with a magnetic stir bar until completely homogeneous. The resulting solution is then transferred into a 500 ml graduated cylinder with a glass stopper. The cylinder is manually and vigorously shaken for 1 minute and placed down on to a bench top for one minute. The volume of the foam layer is then measured in graduated cylinder (foam volume).

### EXAMPLES

The following is a description of the manufacture of compositions of the present invention. Other compositions of the invention can be prepared in an analogous manner by a person of ordinary skill in the art.

### Example 1

The compositions of Table 1 can be manufactured in the following manner:

TABLE 1

| Item | CHEMICAL NAME | | |
|---|---|---|---|
| # | (INCI) | % (W/W) | Supplier |
| 1 | Water | q.s. 100 | |
| 2 | Xanthan Gum | 0.1-5 | Keltrol CG |
| | | | Calgon Corporation |
| | | | Pittsburgh, PA USA |
| 3 | Glycerin | 0.1-30 | Glycerin 00.7% |
| | | | Cognis |
| | | | Cincinnati, OH USA |
| 4 | Disodium EDTA | 0.01-1 | Hamp-ene Na2 |
| | | | Hampshire Chemical |
| | | | Corp. |
| | | | Nashua, NH USA |
| 5 | Kaolin | 0.1-25 | Kaolin |
| | | | Kaopolite, Inc. |
| | | | Union, NJ USA |
| 6 | Titanium | 0.1-5 | Titanium Dioxide |
| | Dioxide | | Sun Chemical Corporation |
| | | | Cincinnati, OH USA |
| 7 | Bentonite | 0.1-25 | Polargel HV BC |
| | | | Whittaker, Clark & |
| | | | Daniels, Inc. |
| | | | South Plainfield, NJ |
| | | | USA |
| 8 | citric Acid | 0.01-2 | Citric Acid Anhydrous |
| | | | Tate & Lyle |
| | | | Decatur, IL USA |
| 9 | Sodium Citrate | 0.01-1 | Sodium Citrate FCC |
| | | | Tate & Lyle |
| 10 | Benzoyl | 0.1-15 | Cadet BPO-78 USP |
| | Peroxide | | Akzo Nobel |
| | | | Chicago, IL USA |
| 11 | Sodium Methyl | 0.1-50 | Tauranol WS |
| | Cocoyl Taurate | | Finetex, Inc. |
| | | | Elmwood Park, NJ |
| 12 | Sodium Lauroyl | 0.1-50 | Hamposyl L-30 |
| | Sarcosinate | | Hampshire Chemical |
| | | | Corp. |
| | | | Nashua NH 03060 |
| 13 | Sodium | 0.1-50 | Mackam HPC-32 |
| | Cocoampho- | | McIntyre Group Ltd |
| | acetate | | University Park IL |
| | | | 60466 |
| 14 | Trideeth-9 | 0.01-5 | NEO-PLC W/S |
| | (and) PEG-5 | | Dragoco |
| | Ethylhexanoate | | Totowa, NJ USA |
| 15 | Menthol | 0.01-2 | Menthol Crystals |
| | | | Technology Flavors |
| | | | and Fragrances |
| | | | Amityville, NY USA |
| 16 | Fragrance | 0.01-2 | Quest International |
| | | | Fragrances USA, Inc. |
| | | | Mount Olive, NJ |

Items 1, 2, and 3 are added into a batch tank and heated and mixed at 55-60° C. for a minimum of 30 minutes. When the mixture is free of lumps, item 4 is added and homogeneously mixed. The batch is then re-circulated, and the temperature is maintained at 45-55° C. Items 5, 6, and 7 are then slowly added to the mixture, and the mixture is mixed at 45-55° C. until the mixture is smooth. Items 8 and 9 are then added to the mixture. The batch is then cooled to 28-30° C. When the temperature of the mixture has cooled to 30° C., item 10 is added and mixed until uniform. Item 10 (“BPO”) is added as a phase, which is prepared by milling BPO with a portion of the water and glycerin until the particle size of the BPO has been reduced to less than 10 μm. Then, item 11, 12, and/or 13 is added during mixing. When the mixture is uniform, item 14, 15, and 16 are premixed and then added to the mixture.

### Example 2

The following three compositions (Formulations A, B, and C) of Table 2 were manufactured in the manner set forth in Example 1.

TABLE 2

| Item | CHEMICAL | | | |
|---|---|---|---|---|
| # | NAME (INCI) | Form. A | Form. B | Form. C |
| 1 | Water | 45.08 | 45.08 | 45.08 |
| 2 | Xanthan Gum | 0.6 | 0.6 | 0.6 |
| 3 | Glycerin | 12.12 | 12.12 | 12.12 |
| 4 | Disodium EDTA | 0.2 | 0.2 | 0.2 |
| 5 | Kaolin | 11 | 11 | 11 |
| 6 | Titanium Dioxide | 3 | 3 | 3 |
| 7 | Bentonite | 9 | 9 | 9 |
| 8 | Citric Acid | 0.4 | 0.4 | 0.4 |
| 9 | Sodium Citrate | 0.3 | 0.3 | 0.3 |
| 10 | Benzoyl Peroxide | 4.5 | 4.5 | 4.5 |
| 11 | Sodium Methyl Cocoyl | 13 | 0 | 0 |
| | Taurate | | | |
| 12 | Sodium Lauroyl | 0 | 13 | 0 |
| | Sarcosinate | | | |
| 13 | Sodium | 0 | 0 | 13 |
| | Cocoamphoacetate | | | |
| 14 | Trideeth-9 (and) PEG- | 0.5 | 0.5 | 0.5 |
| | 5 Ethylhexanoate | | | |
| 15 | Menthol | 0.1 | 0.1 | 0.1 |
| 16 | Fragrance | 0.2 | 0.2 | 0.2 |

### Example 3

Formulations A, B, and C of Example 2 were subjected to the Foaming Test described herein. Formulation A had a foam volume of 450 ml, formulation B had a foam volume of 345 ml, and formulation C had a foam volume of 475 ml.

What is claimed is:

1. A product comprising a composition wherein said composition comprises at least one clay, at least one foaming surfactant, and water and wherein said product comprises instructions directing the user to apply said composition to wet skin and then to remove said composition from the skin.

2. A product of claim 1, wherein said product further comprises alternative instructions directing the user to apply said composition to dry skin, retain the composition on said skin, and to remove said composition from said skin.

3. A product of claim 1, wherein at least one of said at least one foaming surfactant is an anionic surfactant.

4. A product of claim 2, wherein at least one of said at least one foaming surfactant is an anionic surfactant.

5. A product of claim 1, wherein said composition comprises: about 10 to about 50 percent, by weight, of said at least one clay; 10 to about 25 percent, by weight, of said at least one foaming surfactant, and about 20 to about 80 percent, by weight, of said water.

6. A product of claim 2, wherein said composition comprises: about 10 to about 50 percent, by weight, of said at least one clay; 10 to about 25 percent, by weight, of said at least one foaming surfactant, and about 20 to about 80 percent, by weight, of said water.

7. A product of claim 1, wherein said composition has a foam volume of at least 200 ml.

8. A product of claim 2, wherein said composition has a foam volume of at least 200 ml.

9. A product of claim 1, wherein said composition has an anti-acne agent.

10. A product of claim 9, wherein said anti-acne agent is benzoyl peroxide, salicylic acid, or a mixture thereof.

11. A product of claim 2, wherein said composition has an anti-acne agent.

12. A product of claim 11, wherein said anti-acne agent is benzoyl peroxide, salicylic acid, or a mixture thereof.

13. A product of claim 1, wherein said at least one clay is kaolin, bentonite, or a mixture thereof.

14. A product of claim 2, wherein said at least one clay is kaolin, bentonite, or a mixture thereof.

15. A method of promoting a product comprising a composition where said composition comprises at least one clay, at least one foaming surfactant and water, wherein said method comprises directing the user to apply said composition to wet skin and then removing said composition from said skin.

16. A method of claim 15, wherein said product further comprises alternative instructions directing the user to apply said composition to dry skin, retain the composition on said skin, and to remove said composition from said skin.

17. A method of claim 15, wherein at least one of said at least one foaming surfactant is an anionic surfactant.

18. A method of claim 16, wherein at least one of said at least one foaming surfactant is an anionic surfactant.

19. A method of claim 15, wherein said composition comprises: about 10 to about 50 percent, by weight, of said at least one clay; 10 to about 25 percent, by weight, of said at least one foaming surfactant, and about 20 to about 80 percent, by weight, of said water.

20. A method of claim 16, wherein said composition comprises: about 10 to about 50 percent, by weight, of said at least one clay; 10 to about 25 percent, by weight, of said at least one foaming surfactant, and about 20 to about 80 percent, by weight, of said water.

21. A method of claim 15, wherein said composition has a foam volume of at least 200 ml.

22. A method of claim 16, wherein said composition has a foam volume of at least 200 ml.

23. A method of claim 15, wherein said composition has an anti-acne agent.

24. A method of claim 23, wherein said anti-acne agent is benzoyl peroxide, salicylic acid, or a mixture thereof.

25. A method of claim 16, wherein said composition has an anti-acne agent.

26. A method of claim 25, wherein said anti-acne agent is benzoyl peroxide, salicylic acid, or a mixture thereof.

27. A method of claim 15, wherein said at least one clay is kaolin, bentonite, or a mixture thereof.

28. A method of claim 16, wherein said at least one clay is kaolin, bentonite, or a mixture thereof.

29. A method of cleansing the skin, said method comprising:
(i) wetting said skin;
(ii) applying a composition comprising at least one clay, at least one foaming surfactant, and water; and
(iii) removing said composition from said skin.

30. A method of claim 29, wherein at least one of said at least one foaming surfactant is an anionic surfactant.

31. A method of claim 29, wherein said composition comprises: about 10 to about 50 percent, by weight, of said at least one clay; 10 to about 25 percent, by weight, of said at least one foaming surfactant, and about 20 to about 80 percent, by weight, of said water.

32. A method of claim 29, wherein said composition has a foam volume of at least 200 ml.

33. A method of claim 29, wherein said composition has an anti-acne agent.

34. A method of claim 29, wherein said at least one clay is kaolin, bentonite, or a mixture thereof.
